(19) United States
(12) Patent Application Publication
(10) Pub. No.: US 2026/0294432 A1
(43) Pub. Date: Oct. 1, 2026

(54) MATERIAL COMBINATIONS AND PROCESSING METHODS FOR A SURGICAL INSTRUMENT

(71) Applicant: Applied Medical Resources Corporation, Rancho Santa Margarita, CA (US)

(72) Inventors: Andrew Krasts, San Clemente, CA (US); Darci Darnall, Rancho Santa Margarita, CA (US); Eric J. Weiss, San Clemente, CA (US); Timothy M. Hopkins, Rancho Santa Margarita, CA (US)

(21) Appl. No.: 19/704,242

(22) Filed: Jun. 10, 2026

Related U.S. Application Data
(63) Continuation of application No. 19/247,514, filed on Jun. 24, 2025, now Pat. No. 12,672,871.
Application No. 19/247,514 is a continuation of application No. 18/756,597, filed on Jun. 27, 2024, now Pat. No. 12,357,304.
Application No. 18/756,597 is a continuation of application No. 17/514,180, filed on Oct. 29, 2021, now Pat. No. 12,048,431.
(60) Provisional application No. 63/107,321, filed on Oct. 29, 2020.

(51) Int. Cl. A61B 17/072; A61B 17/00

(52) U.S. Cl. CPC A61B 17/072; A61B 2017/00526; A61B 2017/00836; A61B 2017/00845; A61B 2017/00933

(57) ABSTRACT

Surface preparation for sliding surfaces can enhance wear performance for surgical instruments such as surgical staplers which include reusable mechanisms that are used multiple times with single use reload cartridges. To reduce the potential for galling wear in a metal-to-metal sliding engagement, a combination of surface hardening, surface finish, and surface coatings can be applied to metallic components of a surgical instrument. Surface hardening techniques can allow further manufacturing operations such as welding without compromising the strength of the underlying metal substrate. With stainless steel metal substrates, as surface or case hardening techniques can reduce corrosion resistance, a surface coating can be applied to inhibit surface oxidation as well as provide a barrier to metal-to-metal contact. A further lubricious coating layer such as a bone wax coating layer can enhance galling resistance.

### CROSS-REFERENCE TO RELATED APPLICATIONS

This application is a continuation of U.S. patent application Ser. No. 19/247,514 entitled “Material Combinations and Processing Methods for a Surgical Instrument” filed on Jun. 24, 2025, which is a continuation of U.S. patent application Ser. No. 18/756,597 entitled “Material Combinations and Processing Methods for a Surgical Instrument” filed on Jun. 27, 2024, now issued U.S. Pat. No. 12,357,304, which is a continuation of U.S. patent application Ser. No. 17/514,180 entitled “Material Combinations and Processing Methods for a Surgical Instrument” filed on Oct. 29, 2021, now issued U.S. Pat. No. 12,048,431, which claims priority to and benefit of U.S. Provisional Patent Application Ser. No. 63/107,321 entitled “Material Combinations and Processing Methods for a Surgical Instrument” filed on Oct. 29, 2020, each of which is incorporated herein by reference in its entirety.

### BACKGROUND OF THE INVENTION

### Field of the Invention

The present application relates generally to surgical instruments and, more particularly, to material combinations and processing methods for sliding components in end effectors of surgical instruments such as surgical stapling devices.

### Description of the Related Art

Surgical staplers are used to approximate or clamp tissue and to staple the clamped tissue together. As such, surgical staplers have mechanisms to ensure that tissue is properly positioned and captured and to drive staples through the tissue. As a result, this has produced, for example, multiple triggers and handles in conjunction with complex mechanisms to provide proper stapling of the clamped tissue. With these complex mechanisms, surgical staplers can have increased manufacturing burdens, as well as potential sources for device failure and confusion for the user. Thus, reliable stapling of clamped tissue without complex mechanisms is desired.

Surgical staplers can further include replaceable reload cartridges such that multiple cartridges can be used with a stapler in a single surgical procedure. Surgical stapler clamping and firing mechanisms can include metallic components in sliding contact. Further improvements to components that are in sliding contact are desirable to resist wear degradation over multiple firing cycles.

### SUMMARY OF THE INVENTION

In certain embodiments, a surgical stapling instrument is provided herein. The surgical stapling instrument comprises an end effector and a firing member. The end effector comprises a first jaw and a second jaw pivotably coupled to the first jaw. The firing member is longitudinally slidable with respect to the end effector to pivotably move the second jaw with respect to the first jaw and actuate the end effector. At least one of the first jaw, the second jaw, and the firing member comprises: a metallic substrate, a dry film surface coating disposed on the metallic substrate, and a bone wax layer disposed on the dry film surface.

In certain embodiments, a surgical stapler is provided herein. The surgical stapler comprises an elongate shaft and a jaw assembly. The elongate shaft extends from a proximal end to a distal end. The jaw assembly is positioned at the distal end of the elongate shaft. The jaw assembly comprises a cartridge support, an anvil, and a firing member. The cartridge support is configured to receive a reload cartridge having a plurality of staples disposed therein. The cartridge support and the anvil are pivotably movable between an open and a closed configuration. The firing member is longitudinally slidable in engagement with the cartridge support and the anvil in the closed configuration to fire staples. At least one of the anvil, the cartridge support, and the firing member comprises: a case hardened metallic substrate, a dry film surface coating disposed on the metallic substrate, and a bone wax layer disposed on the dry film surface.

In certain embodiments, a method of manufacturing a surgical end effector is provided herein. The method comprises providing a first jaw member, a second jaw member, and a firing member, each comprising a metallic substrate. The method further comprises hardening the metallic substrate of at least one of the first jaw member, the second jaw member, and the firing member to a first predetermined hardness. The method further comprises applying a dry film coating to the hardened at least one of the first jaw member, the second jaw member, and the firing member. The method further comprises applying a bone wax composition to at least one of the first jaw member, the second jaw member, and the firing member.

### BRIEF DESCRIPTION OF THE DRAWINGS

FIG. 1 is a perspective view of an embodiment of surgical stapling device;

FIG. 2 is a perspective view of an embodiment of shaft assembly and jaw assembly for use with the surgical stapling device of FIG. 1;

FIG. 3 is a perspective view of an embodiment of jaw assembly and reload cartridge for use with the surgical stapling device of FIG. 1;

FIG. 4 is a cross sectional perspective view of a proximal end of the jaw assembly of FIG. 3;

FIG. 5 is a cross sectional perspective view of the jaw assembly of FIG. 3;

FIG. 6 is a graph showing an illustrative force versus travel plot for an exemplary embodiment of jaw assembly;

FIG. 7 is a graph showing an illustrative force versus travel plot for another exemplary embodiment of jaw assembly;

FIG. 8 is a graph showing an illustrative force versus travel plot for another exemplary embodiment of jaw assembly;

FIG. 9A is a schematic illustration of a cross section of a metallic substrate for an exemplary embodiment of a component of a surgical stapler;

FIG. 9B is a schematic illustration of a cross section of a metallic substrate for another exemplary embodiment of a component of a surgical stapler; and

FIG. 10 illustrates an exemplary method for preparing a surface of a component of a surgical stapler.

### DETAILED DESCRIPTION OF THE INVENTION

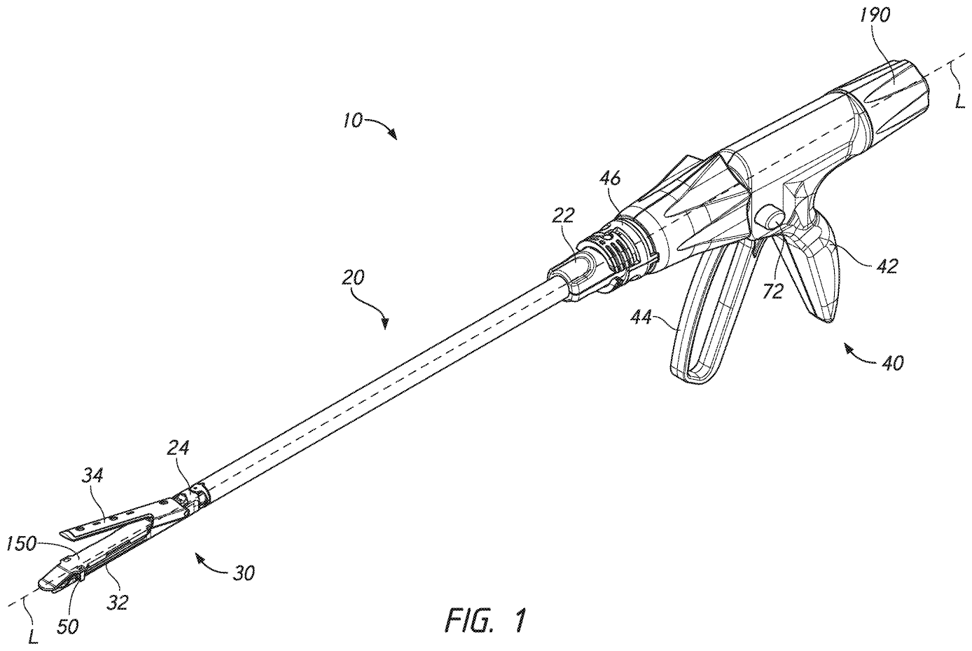

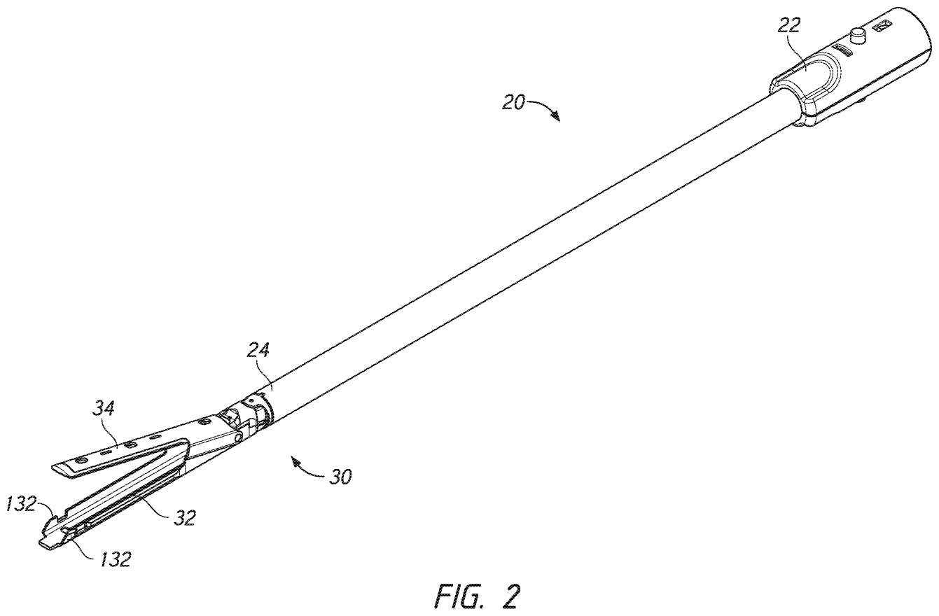

With reference to FIGS. 1-2, embodiments of surgical stapling device are illustrated. The illustrated embodiment of surgical stapler 10 comprises an elongate shaft 20, a jaw assembly 30, and a handle assembly 40. Various aspects of the elongate shaft 20 and jaw assembly 30 described herein can be used interchangeably with either a mechanical handle assembly 40, as illustrated, or a powered handle assembly including, for example, an electric motor. Moreover, it is contemplated that aspects of the elongate shaft 20 and jaw assembly 30 described herein can be used in connection with a shaft assembly configured to be actuated by a robotic surgical system. FIG. 1 illustrates the surgical stapler 10 with the jaw assembly 30 in an open configuration. FIG. 2 illustrates a removable reload shaft assembly comprising the elongate shaft 20 and jaw assembly 30 of the surgical stapler 10 with the jaw assembly 30 in an open configuration.

With continued reference to FIGS. 1 and 2, the illustrated embodiment of surgical stapler 10 can be sized and configured for use in laparoscopic surgical procedures. For example, the elongate shaft 20 and jaw assembly 30 can be sized and configured to be introduced into a surgical field through an access port or trocar cannula. In some embodiments, the elongate shaft 20 and jaw assembly 30 can be sized and configured to be inserted through a trocar cannula having a relatively small working channel diameter, such as, for example, less than 8 mm. In other embodiments, an elongate shaft 20 and jaw assembly 30 can be sized and configured to be inserted through a trocar cannula having a larger working channel diameter, such as, for example, 10 mm, 11 mm, 12 mm, or 15 mm. In other embodiments, it is contemplated that certain aspects of the surgical staplers described herein can be incorporated into a surgical stapling device for use in open surgical procedures.

With continued reference to FIGS. 1 and 2, in the illustrated embodiment, the jaw assembly 30 is coupled to the elongate shaft 20 at the distal end 24 of the elongate shaft 20. The jaw assembly 30 comprises a first jaw 34 pivotally coupled to a second jaw 32. In the embodiment illustrated in FIGS. 1-2, the jaw assembly is articulably coupled to the elongate shaft such that the jaw assembly can be selectively positioned at an articulated position with respect to the central longitudinal axis L. The handle assembly of FIG. 1 includes an articulation knob 190 and articulation mechanism configured to provide continuously selectable articulation of a jaw assembly of an elongate shaft assembly through an articulation range. In an initial configuration, the second jaw 32 includes a plurality of staples positioned within a reload cartridge 50 positioned therein. Thus, the second jaw 32 defines a reload support.

With continued reference to FIGS. 1 and 2, in the illustrated embodiment, the jaw assembly 30 can be actuated from an open configuration (FIG. 1) to a closed configuration to a stapling configuration by an actuation member or beam that is longitudinally slidable within the elongate shaft. In an initial position, the beam can be positioned at the distal end 24 of the elongate shaft 20. With the beam in the initial position, the first jaw 34 is pivoted away from the second jaw 32 such that the jaw assembly 30 is in the open configuration. The actuation beam engages the first jaw 34 upon translation of the actuation member or beam distally along the longitudinal axis L. Translation of the actuation beam distally from the initial position a first distance can actuate the jaw assembly from the open configuration to the closed configuration. With the jaw assembly 30 in the closed configuration, the actuation beam can be returned proximally the first distance to return the jaw assembly 30 to the open configuration. A distal end of the actuation beam can advance a staple slider configured to deploy staples from the second jaw 32 such that further translation of the actuation beam distally past the first distance deploys the plurality of staples 36 from the second jaw 32.

With reference to FIGS. 1-3, in the illustrated embodiment, the handle assembly is coupled to the elongate shaft 20 at the proximal end 22 of the elongate shaft 20. As illustrated, the handle assembly 40 has a pistol grip configuration with a housing defining a stationary handle 42 and a movable handle 44 or trigger pivotably coupled to the stationary handle 42. It is contemplated that in other embodiments, surgical stapler devices including aspects described herein can have handle assemblies with other configurations such as, for example, scissors-grip configurations, or in-line configurations. The handle assembly 40 houses an actuation mechanism configured to selectively advance an actuation shaft responsive to movement of the movable handle 44 to actuate the actuation beam within the elongate a shaft a first distance in an open-close stroke to close the jaw assembly from an initial open position, a second distance beyond the first distance in a firing stroke to fire staples, and to return the actuation beam the second distance and the first distance to an initial position. In certain embodiments, a sliding selector 72 on the handle assembly can allow a user to select whether the handle assembly operates to actuate a jaw assembly in an open-close stroke or a firing stroke. Various embodiments of handle assemblies and associated actuation mechanisms are disclosed in U.S. Pat. No. 9,668,732, entitled “Surgical Stapler Handle Assembly Having Actuation Mechanism With Longitudinally Rotatable Shaft” and U.S. patent application Ser. No. 15/485,620, filed Apr. 12, 2017, entitled “Surgical Stapler Having Articulation Mechanism,” both of which are incorporated by reference herein in their entireties.

With continued reference to FIGS. 1-3, in some embodiments, the surgical stapler 10 can include the plurality of staples positioned in a disposable reload cartridge 50 while the handle assembly 40 and elongate shaft 20 is configured to be reused with multiple staple reload cartridges. In certain embodiments, each reload cartridge 50 can be coupled to a reload cover 150 to shield a tissue contact surface and staple pockets of the reload cartridge before installation to the jaw assembly and that is to be removed before the surgical stapler is introduced to a surgical site. It can be desirable that the handle assembly 40 and elongate shaft 20 can resist wear-related performance degradation such that the stapler can reliably be actuated for multiple clamping and staple firing cycles, each comprising a single use reload cartridge 50. The surgical stapler can include the one or more grasping and firing lockout mechanisms that can limit functionality of the handle assembly to alert a user and enhance patient safety if no reload cartridge is present in the jaw assembly or if a partially or fully fired reload cartridge is present in the jaw assembly. In certain embodiments, a staple deployment member, such as a translatable sled or slider within the reload cartridge 50 can defeat one or more lockout mechanisms when the staple deployment member is in a proximal position in the jaw assembly, corresponding to an unfired reload cartridge is present in the surgical stapler 10.

With reference to FIG. 1, the handle assembly 40 includes a coupler 46 at the distal end thereof. The coupler 46 is adapted to engage the elongate shaft 20 of the surgical stapler 10. The coupler 46 can have a bayonet connection having an outer connector that can removably couple the handle assembly 40 to the elongate shaft 20, and an inner connector that can removably couple the actuation shaft of the handle assembly 42 to the actuation member of the elongate shaft 20. Accordingly, the surgical stapler 10 can be configured such that the handle assembly 40 can be reused with multiple disposable shafts and/or reload cartridges during a surgical procedure. It is contemplated that in other embodiments, the handle assembly and some portion of the elongate shaft can be reusable while a remainder of the elongate shaft and the jaw assembly define a disposable cartridge. In certain other embodiments, the handle assembly and the elongate shaft can be reusable while the jaw assembly defines a disposable cartridge. In still other embodiments, a jaw insert housing a plurality of staples can define a disposable cartridge while the remainder of the surgical stapler is reusable.

As noted above, the shaft assemblies, jaw assemblies, and reload cartridges described herein can also be used in conjunction with a powered stapler handle assembly or an actuator of a robotic surgical system. Various embodiments of powered handle assemblies and associated actuation mechanisms are disclosed in U.S. patent application Ser. No. 15/486,227, filed Apr. 12, 2017, entitled “Reload Shaft Assembly for Surgical Stapler;” U.S. patent application Ser. No. 15/486,008, filed Apr. 12, 2017, entitled “Surgical Stapler Having a Powered Handle;” and U.S. patent application Ser. No. 16/287,748, filed Feb. 27, 2019, entitled “Surgical Stapler Having a Powered Handle;” all of which are incorporated by reference herein in their entireties.

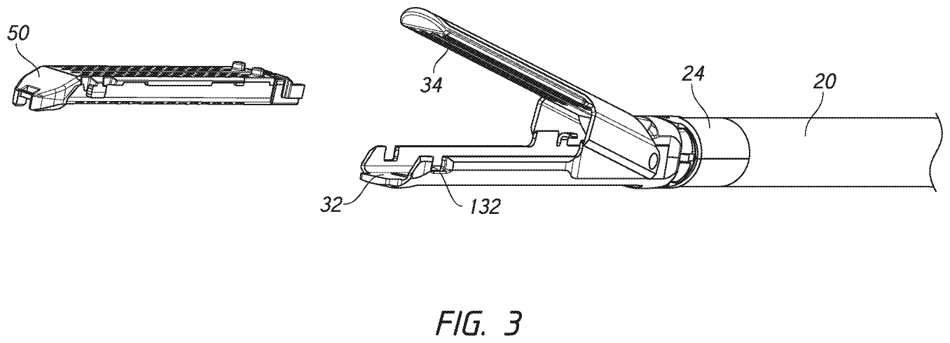

With reference to FIG. 3, a perspective view of the jaw assembly of the elongate shaft 20 is illustrated with the reload cartridge 50 removed from the second jaw 32. As illustrated, the reload cartridge 50 is removably positionable in the reload support defined by the second jaw 32. In the illustrated embodiment, the reload cartridge 50 includes a plurality of staples disposed therein, each staple positioned in its own staple pocket formed through a body of the reload cartridge. An upper surface of the reload cartridge 50 defines a tissue contact surface which, in certain embodiments, can be substantially planar. The reload cartridge further comprises a blade channel formed therein. As illustrated, the blade channel longitudinally extends between rows of staple pockets such that translation of a cutting blade through the blade channel transects tissue between rows of staples that have been deployed into tissue positioned in the jaws when staples are fired. The reload support defined by the second jaw 32 comprises a channel sized and configured to removably receive the reload cartridge 50. For example, in certain embodiments, the channel of the reload support can comprise at least one recess 132 sized and configured to receive a corresponding protruding boss on the reload cartridge 50.

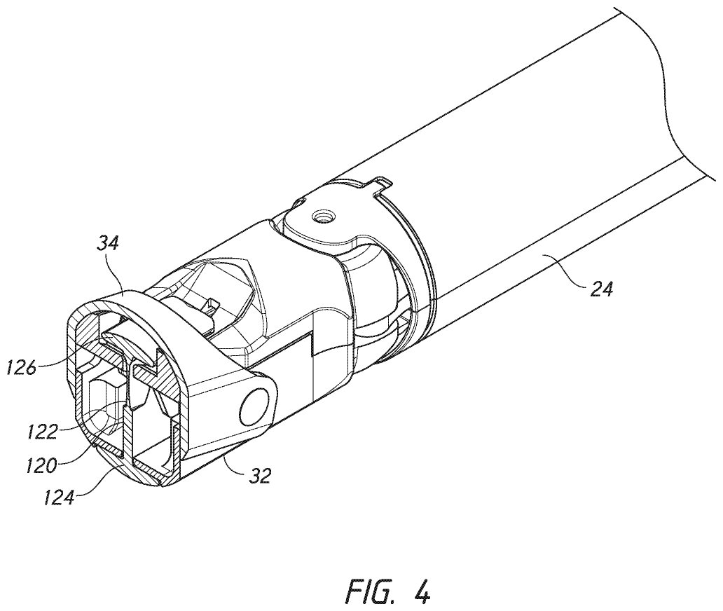

With respect to FIG. 4, an embodiment of jaw assembly is illustrated. A cross section of the jaw assembly has been made generally perpendicularly to a longitudinal axis of the jaw assembly at a proximal end of the jaw assembly just distal the distal end 24 of the elongate shaft to facilitate visibility of certain aspects of operation of the surgical stapler in closure/tissue clamping and firing operations. In the illustrated embodiment, the surgical stapler comprises an actuation mechanism comprising a firing member 120 longitudinally translatable within the first jaw 34 and second jaw 32 of the jaw assembly to actuate the jaw assembly from an open configuration to a closed configuration and subsequently to fire a plurality of staples from a reload cartridge.

With continued reference to FIG. 4, in certain embodiments, the firing member 120 can comprise an I-beam profile with an upper flange 126 connected to a lower flange 124 by a vertical blade member 122. The upper flange 126 is translatable within a channel in the first jaw 34, and the lower flange 124 is translatable within a channel in the second jaw 32. The blade member 122 is translatable within the blade channel of the reload cartridge.

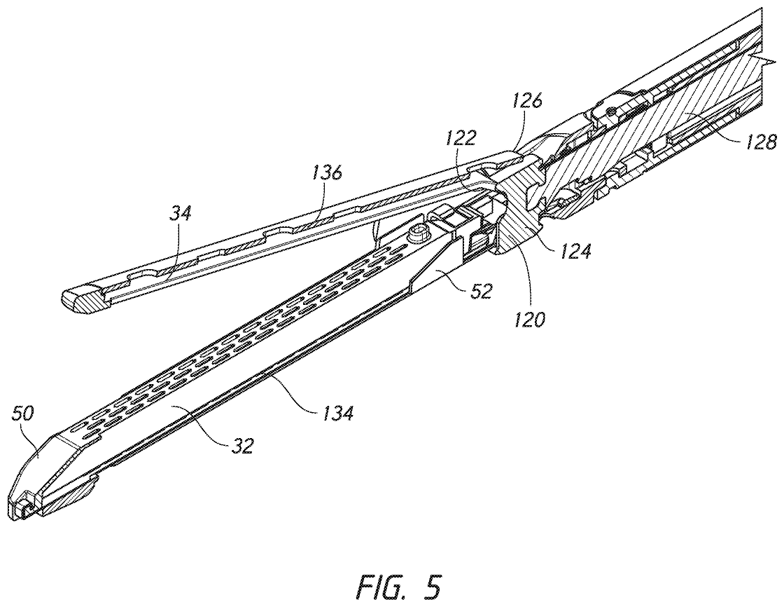

With reference to FIG. 5, certain aspects of an embodiment of jaw assembly of a surgical stapler are illustrated. A cross section of the jaw assembly is illustrated taken longitudinally to further illustrate operation of the actuation mechanism of the surgical stapler. As illustrated, the elongate shaft comprises an actuation member 128 longitudinally slidable therein. The actuation member 128 extends to a distal end to which the firing member 120 is coupled. The reload cartridge 50 can comprise a slider 52 or wedge sled that translates therein, advanced by longitudinally distal movement of the firing member 120 to fire the plurality of staples from the reload cartridge.

With continued reference to FIG. 5, the jaw assembly can comprise an upper channel 136 formed in the first jaw 34. The jaw assembly can further comprise a lower channel 134 formed in the second jaw 32. In the illustrated embodiment, as the firing member 120 is advanced longitudinally to close the jaw assembly and fire staples from the reload cartridge 50, the upper flange 126 slides within the upper channel 136 of the first jaw 34, and the lower flange 124 slides in the lower channel 134 of the second jaw 32. In the illustrated embodiment, a proximal end of the upper channel 136 comprises a ramp such that actuation of the firing member 120 over the ramp pivots the first jaw 34 to a closed configuration. The upper channel 136 further comprises a channel distal the ramp extending such that it is generally parallel to the lower channel 134 with the jaws in the closed configuration.

As it is contemplated that the jaw assembly can be reused with multiple single use reload cartridges 50, it is desirable that sliding surfaces of the firing member 120 and lower and upper channels 134, 136 be configured to minimize performance degradation over multiple use cycles. With tissue clamped between jaws, a lower surface of the upper flange 126 slides against an upper surface of the upper channel 136, and an upper surface of the lower flange 124 slides against a lower surface of the lower channel 134. Accordingly, in certain embodiments, at least these sliding surfaces can be configured to reduce the impacts of frictional engagement therebetween. In other embodiments, it can be desirable that all of the first jaw 34, the second jaw 32, and the firing member 120 be configured to reduce impacts of frictional engagement therebetween.

In certain embodiments, it is desirable that the jaw assembly be configured to be reused with at least ten reload cartridges without significant performance degradation. In certain embodiments can be preferable that the jaw assembly be configured to be reused with at least twelve reload cartridges without significant performance degradation. Furthermore, it is contemplated that it is desirable that the jaw assembly is configured to be operable with a desired number of reload cartridges with an operating load of at least 80 pounds of compressive force applied by first and second jaws 34, 32 to tissue clamped therebetween. In other embodiments, it is desirable that the jaw assembly is configured to be operable with the desired number of reload cartridges with an operating load of at least 100 pounds of compressive force. In still other embodiments, it is desirable that the jaw assembly is configured to be operable with the desired number of reload cartridges with an operating load of at least 120 pounds of compressive force. In certain embodiments, the jaw assembly is configured to be operable with the desired number of reload cartridges at a desired operating load with the jaws misaligned at an angular deviation of up to one degree from the first jaw 34 parallel to the second jaw 32 in a closed configuration. In certain embodiments, the jaw assembly is configured to be operable with the desired number of reload cartridges at a desired operating load with the jaws misaligned at an angular deviation of up to two degrees from the first jaw 34 parallel to the second jaw 32 in a closed configuration. In use, misalignment between the jaws can occur during a stapling operation due to deflection of the jaws in response to the thickness or density of tissue clamped between the jaws.

In general, when two surfaces, such as the lower and upper flanges 124, 126 of the firing member and the respective lower and upper channels 134, 136, are in contact under load and they slide relative to one another, a frictional force opposes the motion. The frictional force is proportional to the load, but ultimately does not depend on the contact area. At the microscopic level, each of the surfaces is not truly flat, but rather has surface irregularities or asperities. These irregularities create localized contact points across which the contact load is distributed. The real contact area is then only a small fraction of the apparent, nominal area. There are multiple types of wear that can either lead to gradual increases in the coefficient of friction When asperities first touch, they deform elastically. However, even small loads when concentrated over a small area can cause large contact stresses that are high enough to cause plastic deformation. The contact points then flatten, forming junctions. Wear in general involves the physical removal of material from a solid object. It can be divided into three categories: abrasive, adhesive and fatigue. Abrasive wear is a more gradual wear process. It occurs when two surfaces rub against one another and the harder surface grinds the softer away. It is often characterized by a rough appearance and can involve the creation of particulate. In many cases, some work hardening (cold working) can occur during this stage. Adhesive wear is a more aggressive form of wear that can lead to galling, especially with metal-to-metal contacting wear surfaces. Highly localized temperatures and the peaks of opposing asperities can deform and move together. Failure to clear debris further exacerbates this type of wear leading to galling with high friction forces.

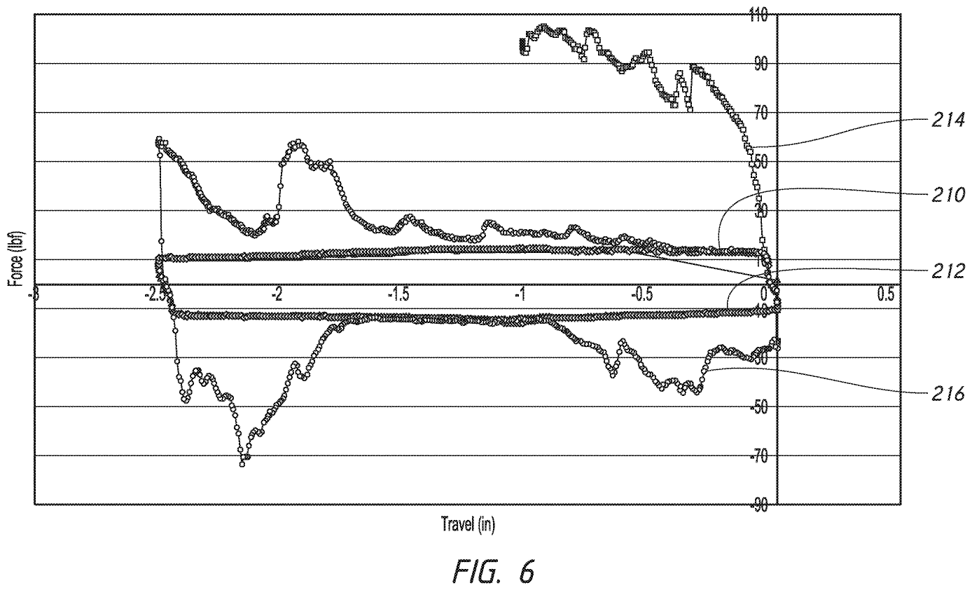

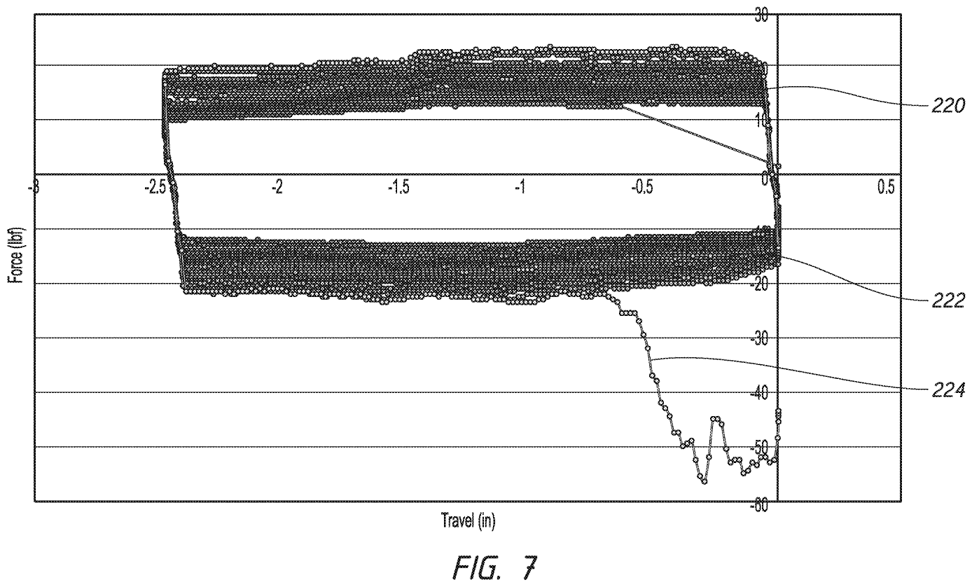

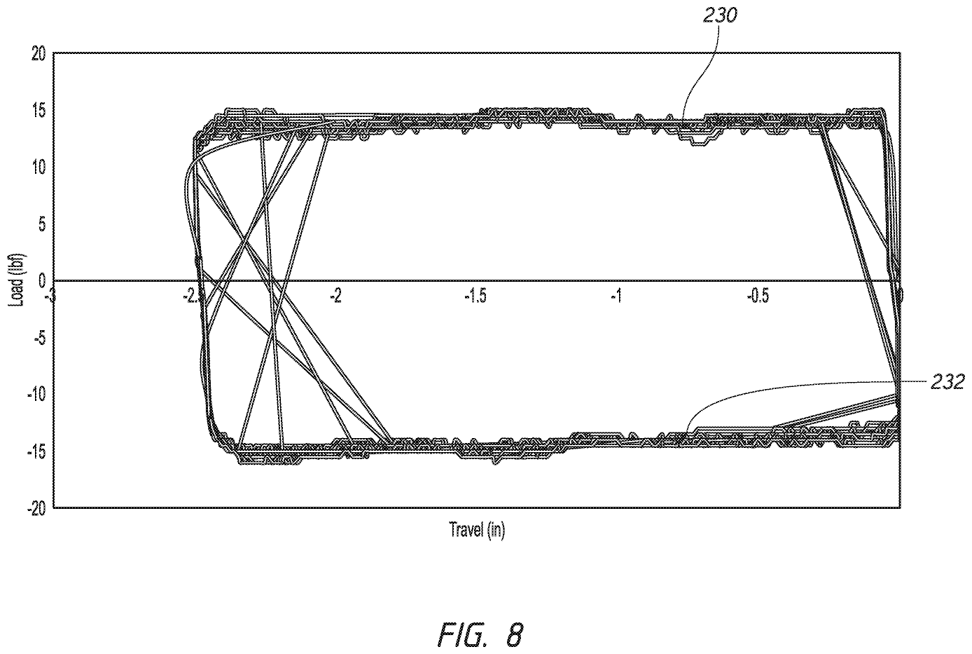

With reference to FIGS. 6-8, certain aspects of sliding engagement between two surfaces are illustrated. Force versus distance plots for exemplary combinations of sliding engagement cycles between a pair of exemplary components are illustrated. FIG. 6 illustrates a force versus distance plot for exemplary components over several sliding engagement cycles illustrating abrasive wear and galling. FIG. 7 illustrates a force versus distance plot for exemplary components over several sliding engagement cycles illustrating a plurality of cycles of abrasive wear followed by galling. FIG. 8 illustrates a force versus distance plot for exemplary components over several sliding engagement cycles illustrating gradual abrasive wear.

With reference to FIG. 6, an illustrative force versus distance plot for several sliding engagement cycles between exemplary sliding components is illustrated. The plot illustrates a progression from at least one sliding engagement cycle with abrasive wear in a first direction 210 and abrasive wear in a second direction 212 to galling wear in a first direction 214 and galling wear in a second direction 216. When the components are moved in the first and second directions with abrasive wear, a friction force between the components is relatively low and relatively constant along a distance traveled. A plot line above the x-axis indicates travel in a first direction (210, 214) while a plot line below the x-axis indicates travel in a second direction (212, 216) opposite the first direction. The height (or depth) of a plot line from the x-axis represents the frictional force required to slide the components. When galling wear is encountered in the first and second directions 214, 216, the frictional force is highly irregular and significantly larger than the frictional force encountered in the abrasive wear regime. Galling is highly detrimental to the function of the device as it increases the coefficient of kinetic friction by a factor greater than one, which as a result multiplies the force required to actuate the device considerably. In some instances, galling can cause the input force required to actuate the device to increase to more than double what it is expected to be for a pair of sliding surfaces. Thus, an embodiment of surgical stapler with sliding components experiencing galling wear after several sliding engagements would be undesirable for use with multiple reload cartridges as the force required to actuate the actuation mechanism would be excessive, stressing components in the shaft assembly and handle assembly, and making the handle assembly difficult to operate.

With reference to FIG. 7 an illustrative force versus distance plot for several sliding engagement cycles between exemplary sliding components is illustrated. The plot illustrates a progression from at least several sliding engagement cycles with abrasive wear in a first direction 220 and abrasive wear in a second direction 222 to galling wear in a second direction 224. When the components are moved in the first and second directions with abrasive wear, a friction force between the components is relatively low and relatively constant along a distance traveled, although the plot line illustrates a plurality of wear cycles with progressively increasing friction forces until galling occurs. In certain embodiments, components exhibiting such wear characteristics can be used in a surgical stapler provided that the components remain in the abrasive wear regime over a sufficient number of wear cycles to permit use of at least a desired number of reload cartridges before experiencing galling wear.

With reference to FIG. 8 an illustrative force versus distance plot for several sliding engagement cycles between exemplary sliding components is illustrated. The plot illustrates repeated operation over a plurality of sliding engagement cycles with abrasive wear in a first direction 230 and abrasive wear in a second direction 232 without the components experiencing galling wear. When the components are moved in the first and second directions with abrasive wear, a friction force between the components is relatively low and relatively constant along a distance traveled with minimal increase from cycle to cycle. It is desirable to configure the jaw assembly for a surgical stapler to have wear characteristics that reduce the likelihood of galling for repeated use with multiple reload cartridges.

In certain embodiments, materials selection for components of a surgical stapler that engage in sliding contact, such as jaw assemblies and firing member can be selected based on certain priorities. For example, as an initial consideration, the materials selected, as well as surface preparation coatings and treatments thereof, are limited to those that meet standards for biocompatibility for use in a patient-contact surgical device. Moreover, it is desirable that the materials selected are able to be joined in a welding operation, which allows flexibility in construction of various aspects of a jaw design, such as by facilitating the use of a two-piece first jaw 34 having a cover or cap that is welded over lower, anvil surface first jaw member forming a channel therebetween. Further, it is desirable that the materials selected have sufficient strength and toughness characteristics for repeated staple firing operations. Additionally, it can be desirable that the materials selected resist oxidation and corrosion. Finally, it is desirable that the materials selected are manufacturable by a variety of processes to promote manufacturing efficiencies, including, for example metal injection molding processes.

Various grades of stainless steel can be selected to achieve the desired characteristics. For example, in certain embodiments, a grade 17-4 stainless steel can be selected for use in sliding components. In other embodiments, a grade 420 stainless steel can be selected for use in sliding components. Grade 420 is a martensitic stainless steel as opposed to a precipitation-hardened stainless steel such as grade 17-4. A grade 420 stainless steel has a relatively high carbon content as compared to a grade 17-4 stainless steel. Thus, desirably, a grade 420 stainless steel is relatively hardenable as compared to a lower-carbon steel. However, martensitic stainless steels tend to be less weldable than precipitation-hardened stainless as brittle martensite can tend to form from rapid cooling of weld zones that can lead to stress induced cracking. Moreover, the relatively higher carbon content of a grade 420 stainless steel can also lead to a relatively lower corrosion resistance. Each of grades 17-4 and 420 is suitable for use in a metal injection molding process.

With regard to material selection, in still other embodiments, a grade 13-8 or a grade 455/465 stainless steel can be selected for use. However, it is noted that these grades of stainless steels tend to be specialty materials, thus these grades of stainless steel may be less desired in view of potential cost, availability, and manufacturability concerns.

As material selection considerations indicate the desirability of metallic, stainless steel materials for use in the jaw assembly and firing member of a surgical stapler, further consideration should be made of preparation and processing the characteristics of these components to reduce the likelihood of galling, which can result from metal-to-metal sliding engagement. In general, materials having a relatively high surface hardness can be more resistant to galling wear. There are various techniques for achieving a relatively high surface hardness in a metallic substrate such as the stainless steel substrates considered for use in sliding components of a surgical stapler. For example, in various embodiments, at least one of: diffusion/thermal-chemical techniques, surface plating techniques, surface coating techniques, and applied energy techniques can be used to prepare a surface of a metallic substrate for use in sliding contact with reduced galling.

In diffusion or thermo-chemical processes a surface layer of a metallic substrate is hardened through the addition of a hardening species such as carbon, nitrogen, or boron at, typically, a relatively high temperature. These processes can be termed ‘case hardening’ in that a goal is to create a relatively hard case or surface layer while maintaining the toughness and ductility of the core. However, typical case hardening techniques have had undesirable consequences on stainless steel materials, especially precipitation hardened stainless steel materials in that typical case hardening techniques have decreased corrosion resistance of a stainless steel material. Moreover, with precipitation hardened stainless steel materials, case hardening methods with relatively high temperatures can result in unintended annealing of the material. Additionally, where a metallic substrate has been formed using a metal injection molding process, the substrate can have relatively high porosity. Thus, the depth of a case hardened layer, absent further modification to the case hardening technique to control a hardened layer depth, may be different from that of a similarly hardened material metallic substrate not formed by a metal injection molding process.

However, certain case hardening techniques can be used on stainless steel materials with fewer or no significant undesirable effects. For example, a relatively low temperature diffusion case hardening technique commercially known under the trademarks S3P (Specialty Stainless Steel Processes) and KOLSTERISING offered by Bodycote plc. This diffusion technique can case harden relatively low carbon stainless steels such as grade 17-4 stainless steels with minimal reductions in corrosion resistance and minimal impact to the underlying strength and ductility of the metallic substrate.

Another technique to creating a hardened layer is through surface modifications to modify the grain structure of the metallic substrate at an outer layer through work hardening. For example, shot peening (impacting the substrate with high velocity shot) or ion implementation (impacting the metallic substrate with high velocity particles) processes can be used to form a hardened surface layer. Advantageously, these processes do not affect surface chemistry and thus should not reduce corrosion resistance. However, if foreign contaminants are present in the shot (for example if shot media is reused), the contaminants can become embedded in the metallic substrate and potentially cause localized sites of reduced corrosion resistance. Moreover, these surface modification processes can present manufacturing challenges as only the surfaces impacted are work hardened, requiring tight control of fixtures, shot size, intensity, and coverage to facilitate consistent results and reduce potential distortion of the substrate.

In certain embodiments, surface plating, that is, introducing a thin layer of metallic compounds on a substrate, can be used to create desired surface hardness properties for sliding components of a surgical stapler. Examples of types of material that can be used in surface plating to provide surface hardness include chrome, electroless nickel, Diamond Like Coating, and ceramics. Advantageously, depending on the material chosen, surface plating can be an immersive process that can be implemented at a component level with consistent surface properties. However, surface plating can affect the weldability of a component as the plating compound will become present in a welded matrix, impacting the strength of the component. Moreover, surface platings, with high hardnesses can also be relatively brittle and can undesirably tend to crack and particulate when heavily loaded in a point contact. In surgical stapler components, in certain instances the engagement of the flanges of the firing member can engage their respective channels at a substantially point contact, especially with misalignment of the jaws or when a large tissue section is clamped between the jaws.

In certain embodiments, a metallic substrate of a sliding component of a surgical stapler can have an applied surface coating to provide desired operation characteristics. However, in general, surface coatings do not adhere as well to a metallic substrate as surface platings. Moreover, similar to surface plating, surface coatings may particulate when loaded in use. Accordingly, it is preferable to use biocompatible surface coating materials. Additionally, surface coatings, if applied before welding a material substrate, may form part of the weld matrix and reduce strength of a welded component. Accordingly, masking a weld site or coating after a welding operation can be selected to minimize the impact of surface coatings on substrate weldability.

Various surface coatings can be applied to a metallic substrate to improve sliding performance. For example, in certain embodiments, lubricants such as those commercially available under the trademark KRYTOX from the Chemours Company, or MOLYKOTE from the Dow Corning Corporation can be applied to sliding surfaces. In other embodiments, a dry film polytetrafluoroethylene (PTFE) coating can be applied to sliding surfaces to increase lubricity therebetween. For example, a coating commercially available as Dry Film RA coating from the Donwell Company, Inc. can be applied to sliding components of a surgical instrument. PTFE dry film materials can be suitable for use in patient contact applications and can be applied strategically by a spraying process or at a component level through an immersive process.

In certain embodiments, bone wax can be applied as a surface coating to improve sliding performance by acting as a lubricant between sliding surfaces. Various bone wax compositions are commercially available and typically comprise primarily beeswax. Bone wax is suitable for use in patient contact applications as it has been traditionally applied to reduce bleeding from bone surfaces during a medical procedure. Desirably, bone wax is tacky and holds well to applied surfaces. Moreover, bone wax typically only undergoes minimal particulation even with point contact engagement between sliding surfaces. However, bone wax can have a relatively low melting transition temperature (it can be approximately 120 F for certain bone wax compositions). Accordingly, consideration of anticipated sterilization and shipping temperature ranges must be assessed to reduce the likelihood of melting and pooling of applied bone wax. Moreover, bone wax is typically manually applied to target surfaces, thus consistent application to recessed surfaces such as jaw assembly channels can require specialized application tools and procedures.

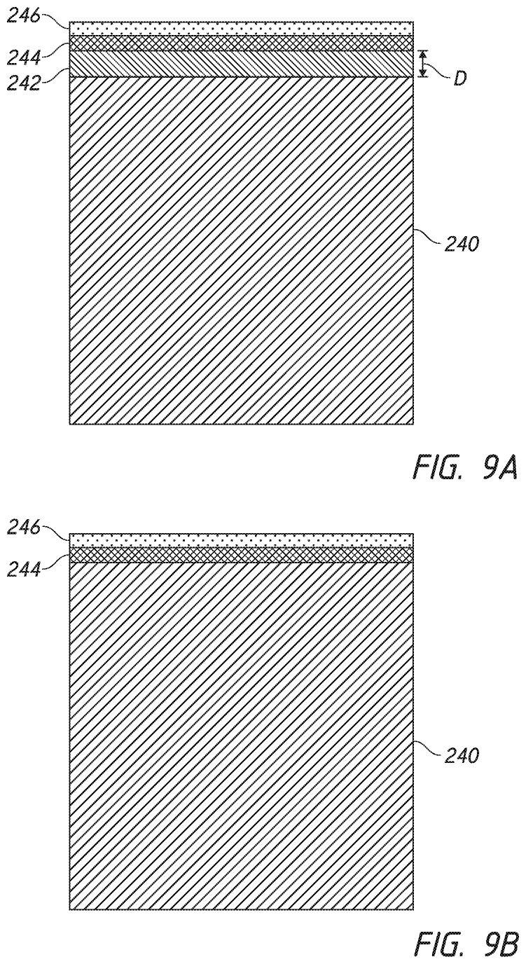

With reference to FIG. 9A, a schematic cross section for a surface of a metallic substrate for use as a sliding surface of a surgical stapling device is illustrated. As discussed above, in view of various considerations for material selection, stainless steel materials are desirable for use in sliding components of a surgical stapling device, such as a firing member and jaw members of a jaw assembly of a surgical stapler. However, in metal-to-metal sliding engagements, these materials can undesirably be subject to galling wear. Accordingly, it is desirable to prepare the sliding surfaces to resist galling. In the illustrated embodiment, the component comprises a metallic substrate 240 having a first strength and a first hardness. A surface layer 242 of the metallic substrate 240 is hardened to a second hardness greater than the first hardness. For example, in certain embodiments, a case hardening method is used to create the surface layer 242 having a depth D at the second hardness. In some embodiments, a diffusion method is used in the case hardening method. In certain embodiments, a low-temperature diffusion case hardening method is used.

With continued reference to FIG. 9A, in certain embodiments, a first surface coating layer 244 can overlie the metallic substrate 240 and a hardened surface layer 242 thereof. For example, in some embodiments, it can be desirable that a first surface coating be selected to limit metal-to-metal contact. Moreover, as certain case hardening methods can tend to reduce corrosion resistance in stainless steel materials, in certain embodiments, it can be desirable that a first surface coating layer can define an oxidation inhibition material.

With continued reference to FIG. 9A, in certain embodiments components of surgical stapler can further comprise a second surface coating layer 246 overlying the first surface coating layer 244. The second surface coating layer 246 can be selected to reduce wear over multiple sliding engagement cycles. For example, in certain embodiments, a bone wax composition can be disposed across sliding surfaces to provide sliding lubrication for multiple reload cycles. Advantageously, a bone wax composition can enhance sliding of components of a surgical stapler in sliding contact even with increases in point contact with inadvertent misalignment.

With continued reference to FIG. 9A, another aspect of surface preparation for sliding components of a surgical stapler is surface finish. For sliding surfaces, it can be undesirable that the surface finish of the sliding components be either relatively smooth, (for example, with a roughness <25 μin) or have a relatively high roughness (for example, with a roughness >75 μin). Sliding surfaces that are highly smooth have a relatively large theoretical contact area, with fewer surface asperities. Thus, these smooth surfaces can tend to facilitate galling and cold welding when in sliding engagement. In contrast, relatively high roughness surfaces can result in relatively high frictional forces and particulation as asperities bind relative to one another. Sliding surfaces having a relatively moderate roughness (for example between about 25 μin and 75 μin) can desirably have a reduced incidence of galling and moderate frictional forces as compared with relatively low roughness surfaces and relatively high roughness surfaces. A moderate roughness can also desirably retain a surface coating when the coated surface is in sliding engagement. In certain embodiments, a moderately rough surface, having a surface roughness between about 25 μin and 75 μin can be prepared through a tumbling process.

With reference to FIG. 9B, a schematic cross section for a surface of another embodiment of metallic substrate for use as a sliding surface of a surgical stapling device is illustrated. Similar to the embodiment of metallic substrate illustrated above with reference to FIG. 9A, the illustrated embodiment of metallic substrate comprises a metallic substrate 240 core, a first coating layer 244, and a second coating layer 246. In certain embodiments, the first coating layer 244 can comprise a dry film, and the second coating layer can comprise a bone wax layer. However, unlike the embodiment of metallic substrate of FIG. 9A, as illustrated in FIG. 9B, the metallic substrate 240 does not comprise a case hardened surface.

In view of the above discussion, various embodiments of material selection and surface preparation to achieve desired sliding performance in a firing member, first jaw, and second jaw without galling over a desired number of firing cycles. In one embodiment, the firing member and jaws of the jaw assembly can comprise a grade 17-4 stainless steel material. The 17-4 stainless steel material can be heat treated to a H900 condition (corresponding to approximately 45 Rockwell C hardness). A surface layer of the material can be case-hardened to approximately 70 Rockwell C hardness. For example, in some embodiments a diffusion method such as an S3P method commercially available from Bodycote plc., can provide a hardened surface layer of approximately between 65 and 70 Rockwell C hardness at a case depth of approximately 25-40 microns. The firing member and jaws of the jaw assembly can be tumbled to a moderate surface roughness. For example, the components can have a surface roughness of between approximately 25 μin and 75 μin. In one embodiment, the components have a surface roughness of approximately 50 μin. A first coating layer of a PTFE dry film can be applied. This first coating layer can inhibit corrosion at the hardened surface and limit metal-to-metal contact. The components can further comprise a second coating layer of a bone wax composition. Advantageously, this combination of materials and processes results in a jaw assembly and firing member that resists galling when repeatedly used with a plurality of single use reload cartridges.

Another embodiment of firing member, first jaw, and second jaw of a surgical stapler comprises a grade 420 stainless steel material that has been heat treated to approximately 55 Rockwell C hardness. The components can be tumbled to a moderate surface roughness. The components can comprise a first coating layer of a PTFE dry film and a second coating layer of a bone wax composition.

Another embodiment of surgical stapler components including at least one of a firing member, a first jaw, and a second jaw comprises a grade 17-4 stainless steel material. The material is heat treated to approximately 45 Rockwell C hardness. For example, as described above, in certain embodiments, the material can be heat treated to an H900 condition. No further case hardening is provided. The components can have a moderately rough surface finish that can be achieved with a tumbling process. The components can comprise a first coating layer of a PTFE dry film and a second coating layer of a bone wax composition. In certain embodiments, one or more of the firing member, first jaw, and second jaw components can be formed of a grade 17-4 stainless steel material with a metal injection molding process. Certain metal injection molding processes can result in a metallic substrate with relatively high porosity relative to a counterpart machined component. Case hardening techniques on such a porous metal injection molded component can create a hardened surface layer having a relatively high depth, but correspondingly relatively high brittleness, which can impact the bulk properties of the case hardened component. Accordingly, embodiments of surgical stapler components without further case hardening processes can be desirable where the components are formed with metal injection molding processes that can create relatively porous metallic substrate.

While in the above embodiments, the components are prepared such that the surface hardnesses of the jaws and the firing members are relatively high (at least about 45 Rockwell C hardness up to about 70 Rockwell C hardness) and substantially identical. It is contemplated that in other embodiments, the firing member can have a slightly lower surface hardness than the jaw members. For example, the firing member can have a surface hardness up to approximately 10 Rockwell C hardness below that of the jaws.

Moreover, while in the above embodiments, case hardening is provided by diffusion or heat treatment, in other embodiments, it is contemplated that shot peening or another work hardening technique can be applied to work harden a surface layer of the metallic substrate of the components. This work hardened surface layer can then be coated with one or more coating layers

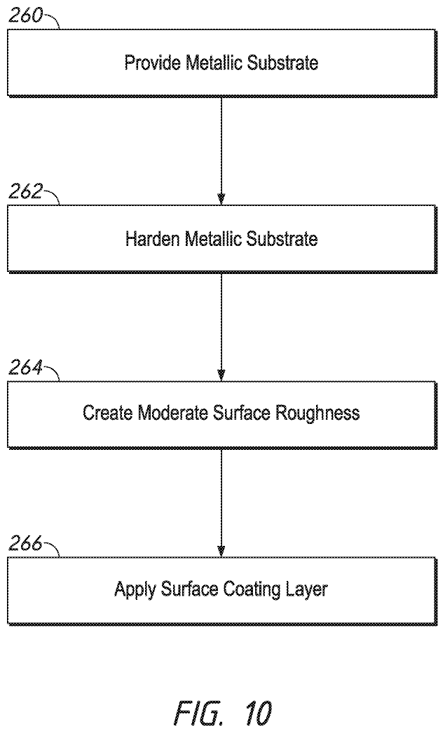

With reference to FIG. 10 a method of preparing a metallic substrate for use as a sliding component of a surgical stapler is illustrated. In certain embodiments, the method can comprise providing a metallic substrate 260. As discussed above, in certain embodiments, the metallic substrate can comprise a stainless steel material such as a grade 17-4 stainless steel or a grade 420 stainless steel. In certain embodiments, providing the metallic substrate can comprise metal injection molding a metallic substrate component.

With continued reference to FIG. 10, the method further comprises hardening the metallic substrate 262 to a desired hardness. In certain embodiments, hardening the metallic substrate can comprise heat treating the metallic substrate. For example, in certain embodiments, hardening the metallic substrate comprises heat treating the metallic substrate to a H900 condition corresponding to a hardness of approximately 45 HRC. In various embodiments, hardening the metallic substrate can comprise case hardening the metallic substrate with a diffusion process. In other embodiments, hardening the metallic substrate can comprise work hardening the surface layer of the metallic substrate such as by shot peening. In certain embodiments, such as schematically illustrated in FIG. 9B, the metallic substrate is hardened to a hardness of approximately 45 HRC by heat treatment without further case hardening. In other embodiments, such as schematically illustrated in FIG. 9A, the metallic substrate is hardened with a heat treatment process and subsequently case hardened such as with a diffusion process to achieve a relatively high surface hardness. In some embodiments, the surface layer can be hardened to between about 45 HRC and 75 HRC. It can be desirable that the surface layer is hardened to at least about 55 HRC. In certain embodiments, the surface layer is hardened to about 70 HRC. In certain embodiments, a metallic substrate to form firing member components for a surgical stapler is surface hardened to a first hardness, and a metallic substrate to form a first jaw and a second jaw is surface hardened to a second hardness different from the first hardness. In certain embodiments, the second hardness is within approximately 10 HRC greater than the first hardness.

With continued reference to FIG. 10, in certain embodiments, the method further comprises providing a moderate roughness surface finish 264. In certain embodiments providing a moderate roughness surface finish can comprise tumbling the components. In certain embodiments, the moderate roughness surface finish can comprise a surface roughness between approximately 25 μin and 75 μin. In some embodiments, the surface roughness is approximately 50 μin.

With continued reference to FIG. 10, in certain embodiments, the method further comprises applying at least one surface coating 266. In certain embodiments, applying at least one surface coating comprises applying a first surface coating and applying a second surface coating. In certain embodiments, applying at least one surface coating comprises applying a PTFE dry film and applying a bone wax composition.

Although this application discloses certain preferred embodiments and examples, it will be understood by those skilled in the art that the present inventions extend beyond the specifically disclosed embodiments to other alternative embodiments and/or uses of the invention and obvious modifications and equivalents thereof. Further, the various features of these inventions can be used alone, or in combination with other features of these inventions other than as expressly described above. Thus, it is intended that the scope of the present inventions herein disclosed should not be limited by the particular disclosed embodiments described above, but should be determined only by a fair reading of the claims which follow.

What is claimed is:

1. A surgical stapler comprising:
a jaw assembly; and
a firing member longitudinally slidably engaged with the jaw assembly,
wherein the jaw assembly comprises a first sliding surface and the firing member comprises a second sliding surface in sliding engagement with the first sliding surface; and
wherein the first sliding surface and the second sliding surface each comprise:
a metallic substrate;
a hardened surface layer defining an outer surface of the metallic substrate;
an oxidation reduction layer overlying the hardened surface layer; and
a lubricant layer overlying the first surface coating layer.

2. The surgical stapler of claim 1, wherein the metallic substrate comprises a stainless steel material.

3. The surgical stapler of claim 2, wherein the stainless steel material comprises one of a grade 17-4 stainless steel and a grade 420 stainless steel.

4. The surgical stapler of claim 1, wherein the hardened surface layer is case hardened to a predetermined depth.

5. The surgical stapler of claim 1, wherein the hardened surface layer is work hardened.

6. The surgical stapler of claim 1, wherein the hardened surface layer comprises a surface plating layer.

7. The surgical stapler of claim 1, further comprising at least one reload cartridge receivable in the jaw assembly, the reload cartridge comprising a plurality of staples deployable therefrom upon longitudinal sliding of the firing member relative to the jaw assembly.

8. The surgical stapler of claim 7, wherein the at least one reload cartridge comprises a plurality of reload cartridges and wherein the first sliding surface and the second sliding surface are configured such that the jaw assembly and firing member are operable to deploy staples from all of the plurality of reload cartridges without galling wear between the first sliding surface and the second sliding surface.

9. The surgical stapler of claim 8, wherein the plurality of reload cartridges comprises at least ten reload cartridges.

10. The surgical stapler of claim 8, wherein the plurality of reload cartridges comprises at least twelve reload cartridges.

11. The surgical stapler of claim 8, wherein the jaw assembly is operable with an operating load of at least 80 pounds of compressive force.
